(19) United States
(12) Patent Application Publication
(10) Pub. No.: US 2010/0152825 A1
(43) Pub. Date: Jun. 17, 2010

(54) Absorbable Pacing Lead Assemblies

(76) Inventor: Norman H. Schulman, Glen Cove, NY (US)

Correspondence Address: RISSMAN HENDRICKS & OLIVERIO, LLP, 100 Cambridge Street, Suite 2101, BOSTON, MA, 02114, US

(21) Appl. No.: 12/616,089

(22) Filed: Nov. 10, 2009

Related U.S. Application Data
(60) Provisional application No. 61/112,960, filed on Nov. 10, 2008.

(51) Int. Cl. A61N 1/05

(52) U.S. Cl. 607/119

(57) ABSTRACT

An absorbable pacing lead assembly may comprise a core, a conductive coating surrounding the core, and an insulator surrounding the conductive coating along a middle portion of the assembly. In some aspects, a distal end of the assembly may comprise a barb.

### TECHNICAL FIELD

The present disclosure relates generally to temporary pacing lead assemblies for delivering electrical stimulation pulses to an organ and, more particularly, absorbable pacing lead assemblies.

### BACKGROUND

Arrhythmias can occur following cardiac surgery such as open-heart surgery. When a patient's heart is not in sinus rhythm, cardiac output is not optimal, putting the patient's life at risk.

Temporary pacing leads are often attached to the myocardium at the end of the cardiac surgical procedure. Once the leads are placed and connected to an external pacing device, the electrical activity of the heart can be sensed and paced when necessary.

Temporary pacing leads can be placed on the right atrium, the right ventricle, or both. When only the atrium or the ventricle is paced, this is referred to as single-chamber pacing. Single-chamber pacing almost invariably refers to placing the temporary pacing lead on the right ventricle to ensure that the ventricles adequately pump blood through the body. However, if the ventricles are not synchronized with the atria, or if the atria are not pumping as effectively as they should, this often results in hypotension or low cardiac output. Therefore, ventricular or single-chamber pacing alone does not always result in optimal cardiac output.

Pacing of both chambers is referred to as dual-chamber pacing. Dual-chamber pacing ensures that the atria and ventricles are synchronized. As a result, cardiac output is increased with no appreciable increase in workload to the ventricle. Although temporary pacing lead placement on the atrium may be more difficult, atrial pacing can be a lifesaving measure, especially when ventricular pacing is ineffective

Some conventional cardiac pacing lead assemblies have comprised an electrode and a flexible insulated conductor for connecting the electrode to a cardiac stimulator. The electrodes are placed directly into tissue in direct contact with heart muscle, overlying the atria and ventricles. If the pacing leads and electrodes are to be subsequently removed, as is the case when a patient only requires cardiac pacing following an operation, it is desirable that the electrodes can be removed with minimal risk of damage to the wall of the heart.

If it is determined that a patient no longer needs cardiac stimulation within three days after cardiac surgery, the pacing lead and electrode can typically be removed. However, if it is determined that the pacing lead and electrode need to remain in place more than three days after cardiac surgery, the pacing lead and electrode may never be removed. Instead, the lead and electrode are left in place and cut off so as not to extend from the patient's body. In such instances, the pacing leads and electrodes, typically comprising insulated wires, sometimes lead to infection. According to some studies, infection arises in about 2% of the over 600,000 per year open heart surgeries that require post-surgery pacing.

It may be desirable to provide the pacemaker industry with absorbable pacing lead assemblies. The absorbable pacing lead assemblies are capable of conducting low voltage electricity for pacing and may be left in a patient's body after cardiac surgery.

### SUMMARY OF THE INVENTION

According to various aspects of the disclosure, an absorbable pacing lead assembly may comprise a core, a conductive coating surrounding the core, and an insulator surrounding the conductive coating along a middle portion of the assembly. In some aspects, a distal end of the assembly may comprise a barb.

In accordance with various aspects of the disclosure, an absorbable pacing lead assembly may comprise a conductive core, an insulator surrounding the conductive core, and a coating surrounding the insulator at a distal end of the assembly. The insulator may include at least one fenestration providing electrical conductivity between the core and the coating. In some aspects, the distal end of the assembly may comprise a barb.

### BRIEF DESCRIPTION OF THE DRAWINGS

FIG. 1 is a schematic illustration of an exemplary absorbable pacing lead assembly in accordance with various aspects of the disclosure.

FIG. 2 is a schematic illustration of an exemplary absorbable pacing lead assembly in accordance with various aspects of the disclosure.

### DETAILED DESCRIPTION

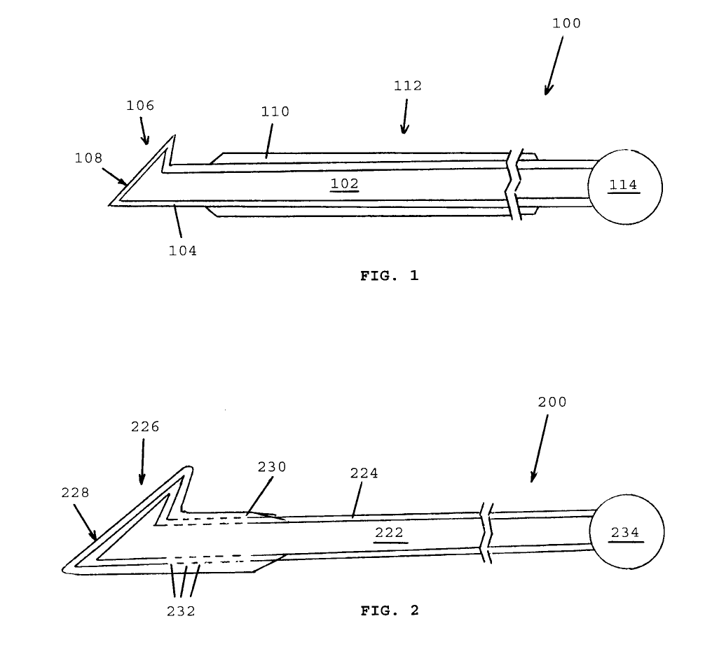

Referring now to the drawings in greater detail, FIG. 1 illustrates an exemplary absorbable pacing lead assembly in accordance with various aspects of the present disclosure. The absorbable pacing lead assembly 100 comprises a core 102 surrounded by a conductive coating 104 along at least a portion of a length of the assembly 100. A distal end 106 of the lead assembly 100 may include a barb 108, which is sufficiently rigid to facilitate tissue fixation with the wall of the heart. The pacing lead assembly 100 may include an insulator 110 surrounding the conductive coating 104 along a middle portion 112 of the assembly 100. The conductive coating 104 may be electrically coupled to an electric source 114 such as, for example, a pacemaker. The electric source 114 may comprise, for example, a low voltage battery. In some aspects, the low voltage battery may comprise a 9 volt battery.

According to various aspects, the core 102 may comprise a conventional solid suture. In some aspects, the core may comprise a medium absorbable rate suture such as, for example, poliglecaprone. The poliglecaprone may comprise poliglecaprone 25. In some aspects, the core 102 may comprise a slower absorbable rate suture such as polydioxanone.

In accordance with various aspects of the disclosure, the conductive coating 104 may comprise, for example, a ferrous sulfate (FeSO₄) compound, and the insulator may comprise poliglecaprone or polydioxanone.

Referring now to FIG. 2, an exemplary absorbable pacing lead assembly in accordance with various aspects of the present disclosure is illustrated. The absorbable pacing lead assembly 200 comprises a conductive core 222 surrounded by an insulator 224 along at least a portion of a length of the assembly 200. A distal end 226 of the lead assembly 200 may include a barb 228, which is sufficiently rigid to facilitate tissue fixation with the wall of the heart. The distal end 226 of the pacing lead assembly 200 may include a coating 230 surrounding the insulator 224 of the assembly 100. The insulator 224 may include one or more fenestrations 232 at the distal end 226 of the pacing lead assembly 200. The conductive core 222 may be electrically coupled to an electric source 234 such as, for example, a pacemaker. The electric source 234 may comprise, for example, a low voltage battery. In some aspects, the low voltage battery may comprise a 9 volt battery.

According to various aspects, the conductive core 222 may comprise, for example, a ferrous sulfate (FeSO₄) gel. The insulator 224 may comprise, for example, an absorbable stitch comprising a material such as, for example, poliglecaprone or polydioxanone.

The coating 230 may comprise, for example, a ferrous sulfate (FeSO₄) compound. The fenestrations 232 may comprise micro-sized fenestrations that permit low voltage electrical conductivity between the coating 230 and the conductive core 222.

It should be appreciated that the insulators 110, 224 may be comprised of other absorbable materials, including absorbable suture materials. The material for constructing the insulators 110, 224 may be selected so as to facilitate absorption of the insulators 110, 224 in about three months. In some aspects, the insulators 110, 224 may be constructed of material such as, for example, polyglycolic acid, which may be absorbed in about two weeks. In any event, the material of the insulators 110, 224 may be chosen to provide any absorption time desired by a physician.

In use, in post-operative open heart surgery, an absorbable pacing lead assembly 100, 200 may be fixed to body tissue of a patient, such as, for example, the wall of the heart, via a barb 108, 228 at the distal end 106, 226 of the assembly 100, 200. The absorbable pacing lead assembly 100, 200 conducts the low voltage electricity necessary for pacing from the electric source 234 to the heart. The pacing lead assembly 100, 200 may be removed from the patient within the first few days after the cardiac procedure, if it is determined that the removal can be done without damaging the heart tissue. If the pacing lead assembly 100, 200 is left attached to the heart tissue for more than a few days in order to ensure the patient's well-being, it may be determined that the pacing lead assembly 100, 200 cannot be safely removed. The absorbable pacing lead assembly 100, 200 may be left in place and cut off so as not to extend from the patient's body. After an amount of time determined by the material comprising the insulator 110, 224, the pacing lead assembly 100, 200 will be absorbed by the patient's body, thereby reducing the risk of infection due to the pacing lead assembly 100, 200 left in the body.

It will be apparent to those skilled in the art that various modifications and variations can be made to the pacing lead assemblies of the present disclosure without departing from the scope of the invention. Other embodiments of the invention will be apparent to those skilled in the art from consideration of the specification and practice of the invention disclosed herein. It is intended that the specification and examples be considered as exemplary only.

What is claimed is:

1. An absorbable pacing lead assembly, comprising:
a core;
a conductive coating surrounding the core; and
an insulator surrounding the conductive coating along a middle portion of the assembly.

2. The assembly of claim 1, wherein a distal end of the assembly comprises a barb.

3. An absorbable pacing lead assembly, comprising:
a conductive core;
an insulator surrounding the conductive core;
a coating surrounding the insulator at a distal end of the assembly, the insulator including at least one fenestration providing electrical conductivity between the core and the coating.

4. The assembly of claim 3, wherein the distal end of the assembly comprises a barb.
